(19) United States
(12) Patent Application Publication
(10) Pub. No.: US 2009/0299644 A1
(43) Pub. Date: Dec. 3, 2009

(54) SYSTEM AND METHOD FOR SELECTING SAMPLES FROM AN ANTIBODY ANALYSIS FOR DISPLAY OF COMPLETE SPECIFICITY ANALYSIS DATA

(75) Inventors: Samuel Leah, Wirral (GB); Andrew Canterbury, Cheshire (GB); Erin McCombs, Oregon, WI (US); Donald Munroe, Chaska, MN (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/363,570

(22) Filed: Jan. 30, 2009

Related U.S. Application Data
(60) Provisional application No. 61/025,740, filed on Feb. 1, 2008.

(51) Int. Cl. G01N 33/48

(52) U.S. Cl. 702/19

(57) ABSTRACT

A system and method provides for selecting one or more samples from an antibody specificity analysis for displaying complete antibody specificity analysis data. A plurality of sample sera are run through a protein panel for antibody analysis and a specificity analysis is completed. A positive or negative screening result is displayed for each sample within the panel, and for each positive sample, preliminary diagnostic data such as the PRA value and intensity value are provided. The preliminary diagnostic data is then reviewed by a user to determine whether complete specificity data of each positive sample is needed. In one embodiment, the complete specificity data is available for purchase on a sample-by-sample basis so that if only a small amount of samples are deemed useful, purchase of the complete specificity data for an entire panel of samples is avoided. The specificity data is encrypted until the user decides to purchase it.

### CROSS-REFERENCE TO RELATED APPLICATIONS

The present application claims the benefit of the filing date of U.S. Provisional Application No. 61/025,740 filed Feb. 1, 2008, the disclosure of which is incorporated by reference herein in its entirety.

### BACKGROUND OF THE INVENTION

1. Field of the Invention

The present invention relates to a system, method and computer program product for selecting one or more samples from a laboratory analysis test for displaying complete analysis data, and more specifically to selecting one or more samples from an antibody specificity analysis for displaying complete antibody specificity analysis data.

2. Background of the Invention

Approximately 75 percent of sera sent for antibody analysis are negative—that is, they do not show the presence of antibodies against a panel of cells or human leukocyte antigens (“HLA”). Currently, the most effective method for determining the presence and specificity of antibody is to initially screen sera with a pooled screening kit. FIG. 1 is a flowchart illustration of an antibody analysis workflow that begins with pooled screening (step 102). Pooled screening provides a simple “yes” or “no” answer as to whether antibody is present in the sera. If antibody is present (stage 104), a second, more detailed analysis (stage 106) is needed to determine a panel reactive antibody (“PRA”) value and a specificity. The PRA value is the percentage of cells from a panel of blood donors against which a potential recipient's serum reacts, and is essentially a measure of a patient's level of sensitization to donor antigens. Specificity is the apparent HLA antigen(s) to which the antibodies in a patient sera may be directed. The ability of the antibody or polypeptide to bind to a known binding partner. Examples of binding partners include but are not limited to antibodies, functional fragments of antibodies, enzymes, functional fragments of enzymes. In this secondary analysis (stage 106), a new protein panel is needed, and so the cost of new reagents, the time to complete another test, and the additional sera required make the process exceedingly inefficient. If the secondary analysis still fails to define specificity (stage 108), a third, single antigen test (stage 110) is required to provide high resolution specificity analysis. This third test again adds to the cost and time to complete the test, as well as the required amount of sera from a patient.

Attempts to limit the number of tests require have been developed. One example is the DYNACHIP™ Antibody Analysis System (Invitrogen, Carlsbad, Calif.). In the DYNACHIP™ system, a single assay analyzes PRA and specificities, eliminating the need for multiple tests. However, the DYNACHIP™ system still requires the use of an entire panel, which increases the costs required for testing

Thus, it is desired to develop systems and methods to more efficiently determine specificity in HLA typing and antibody analysis so as to save the time, reduce costs and minimize the amount of sera required for an antibody analysis.

### SUMMARY OF THE INVENTION

The present invention relates to systems and methods for selecting one or more samples from an HLA typing analysis for displaying complete antibody specificity analysis data. In one embodiment, sera samples may be run through a protein panel for antibody analysis and a full specificity analysis may be completed. A positive or negative screening result may be displayed for each sample tested, and along with each positive sample, preliminary diagnostic data such as the panel reactive antibody (“PRA”) value and intensity value may be provided. A user may then review the preliminary diagnostic data to determine whether complete specificity data of each positive sample is needed, and may select at least one positive sample to obtain the complete specificity data. In one embodiment, the specificity data is available for purchase on a sample-by-sample basis so that if specificity data is desired for a small amount of samples, purchase of the specificity data for the entire panel of samples is avoided. In another embodiment, the specificity data may be encrypted until the user decides to purchase it.

The present invention also relates to a method for selecting one or more samples for displaying complete antibody specificity analysis data, the method comprising: receiving complete specificity analysis data; displaying an initial screening result for each sample, wherein the screening result is negative or positive, and wherein the positive sample may include preliminary diagnostic data, the preliminary diagnostic data being derived from the specificity analysis; selecting at least one of the positive samples to display complete specificity analysis data, wherein the selection may be based upon the preliminary diagnostic data; and displaying the complete specificity analysis data for the selected samples.

In another aspect of the invention, a system for selecting one or more samples for displaying complete antibody specificity analysis data comprises: receiving means for receiving complete specificity analysis data; display means for displaying a screening result for each sample, the screening result being positive or negative, and wherein the positive sample includes preliminary diagnostic data, the preliminary diagnostic data being derived from the specificity analysis; selection means for selecting at least one of the positive samples to display complete specificity analysis data, wherein the selection is based upon the preliminary diagnostic data; and; display means for displaying the complete specificity analysis data for the selected samples.

In a further embodiment of the invention, a computer program product embodied on a computer-readable medium comprises computer code for selecting one or more samples for displaying complete antibody specificity analysis data, wherein the computer code may be operable for: receiving a complete specificity analysis; determining a screening result for each sample, the screening result being positive or negative, and wherein the positive sample includes preliminary diagnostic data, the preliminary diagnostic data being derived from the specificity analysis; displaying an interactive screening report to a user, wherein a user can select at least one of the positive samples for displaying complete specificity analysis data; and displaying the complete specificity analysis data for the selected samples.

Additional aspects related to the invention will be set forth in part in the description which follows, and in part will be obvious from the description, or may be learned by practice of the invention. Aspects of the invention may be realized and attained by means of the elements and combinations of various elements and aspects particularly pointed out in the following detailed description and the appended claims.

It is to be understood that both the foregoing and the following descriptions are exemplary and explanatory only and are not intended to limit the claimed invention or application thereof in any manner whatsoever.

### BRIEF DESCRIPTION OF THE DRAWINGS

The accompanying drawings, which are incorporated in and constitute a part of this specification exemplify the embodiments of the present invention and, together with the description, serve to explain and illustrate principles of the inventive technique. Specifically:

FIG. 1 is a flow chart illustration of a currently known workflow for antibody analysis which requires multiple stages of testing;

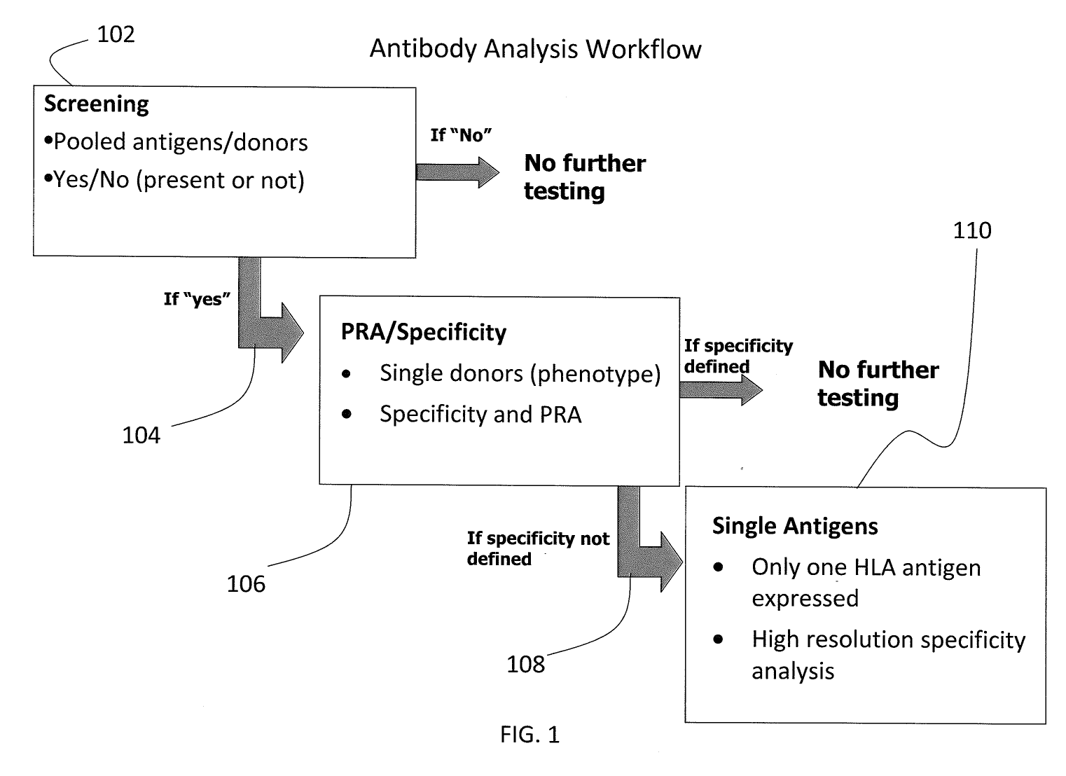

FIG. 2 is an image of a graphical user interface for a computer program product embodying a system for separating and screening preliminary diagnostic data, according to one embodiment of the invention;

FIG. 3 depicts a workflow for a system and method for encrypting specificity data during the antibody analysis; and

FIG. 4 depicts a method for separating and screening preliminary diagnostic data according to one embodiment of the invention.

FIG. 5 comparison of results of 39 CDC-XMs with those of DYNACHIP™ tests with closely matched phenotypes yielded mean DYNACHIP™ intensity values for positive and negative CDC-XMs of 0.547 and 0.252, respectively (p=0.00003).

### DETAILED DESCRIPTION OF THE INVENTION

In the following detailed description, reference will be made to the accompanying drawing(s), in which identical functional elements are designated with like numerals. The aforementioned accompanying drawings show by way of illustration and not by way of limitation, specific embodiments and implementations consistent with principles of the present invention. These implementations are described in sufficient detail to enable those skilled in the art to practice the invention and it is to be understood that other implementations may be utilized and that structural changes and/or substitutions of various elements may be made without departing from the scope and spirit of present invention. The following detailed description is, therefore, not to be construed in a limited sense. Additionally, the various embodiments of the invention as described may be implemented in the form of software running on a general purpose computer, in the form of a specialized hardware, or combination of software and hardware.

The methods described provide a system and method for selecting one or more samples from an antibody specificity analysis for displaying complete antibody specificity analysis data. A plurality of sample sera are run through a protein panel for antibody analysis and a specificity analysis is completed. A positive or negative screening result is displayed for each sample within the panel, and for each positive sample, preliminary diagnostic data such as the panel reactive antibody (“PRA”) value and intensity value are provided. The preliminary diagnostic data is then reviewed by a user to determine whether complete specificity data of each positive sample is needed. In one embodiment, the complete specificity data is available for purchase on a sample-by-sample basis so that if only a small amount of samples are deemed useful, purchase of the complete specificity data for an entire panel of samples is avoided. The specificity data may be encrypted until the user decides to purchase it.

Although the example embodiment described herein is an antibody analysis system, one skilled in the art will appreciate that the invention is applicable to any type of laboratory diagnostic testing where a panel of results is provided for analysis, such as sequence-based typing.

The invention saves time and money for a user performing an antibody analysis, since the user can review preliminary diagnostic data for each sample first to determine if complete specificity data is needed. Instead of purchasing an entire panel of specificity data and sorting through the complete specificity data for each of the samples, the user can first determine which samples may be useful for further review and only purchase those that are potentially valuable. Additionally, when combined with an array processor such as a DYNACHIP™ Antibody Analysis System (Life Technologies; Carlsbad, Calif.), only a small volume of sera is needed for the selecting of one or more samples from an antibody specificity analysis for displaying complete antibody specificity analysis data.

The following embodiment describes the use of the DYNACHIP™ Antibody Analysis System for performing automated processing of serum samples including the steps of dispensing, incubating, washing, image detection and results analysis. The DYNACHIP™ Antibody Analysis System provides the complete specificity data used by the methods for selecting and displaying the complete specificity data of certain samples.

In one embodiment of the invention, a user with the DYNACHIP™ System runs an initial analysis of a serum on a protein panel that is displayed as a screening product. It is important to note that while the initial analysis is a screen, a full specificity analysis is completed for each sample in the panel, although it is not displayed at the moment. The system is then configured to display the result of the analysis as a positive or negative screening result. For a positive result, the system will display statistics from the specificity analysis, known as preliminary diagnostic data. The user can then review the preliminary diagnostic data of each positive sample to determine whether to view the complete specificity data of a particular sample. When the user selects one or more samples, the system reveals the complete specificity data to the user for full review of the selected samples. Therefore, the user does not need to sort through the complete specificity data for all samples to determine which are useful, as the system has accomplished this.

In another embodiment, the complete specificity data for each sample is only available by purchasing the sample. However, instead of purchasing the data for all samples, when only a portion of the samples may be useful, the user can purchase the specificity data of only the samples that are believed to be useful, as determined from the preliminary diagnostic data. The user can purchase as little as 1 sample or all of the samples, depending on how potentially useful each individual sample is believed to be. Therefore, the preliminary diagnostic data is particularly important to help a user purchase only the specificity data that is most helpful for a particular application.

To simplify the purchase process, in one embodiment, the user is provided with “specificity credits” to apply toward the purchase of the specificity data. For each panel, the user may initially be given 10 or 25 specificity credits to apply towards a purchase of 10 or 25 samples. If the user determines that additional samples may be useful, additional specificity credits can be purchased.

In one embodiment, the preliminary diagnostic data includes a panel reactive antibody (“PRA”) value and an intensity value. In addition, the values of the preliminary diagnostic data can be customized by the user to help select the most useful samples from a panel. For example, the user can adjust the intensity value to require a minimum and maximum signal, for example to eliminate positive results that could be from background. In this manner, the user is able to make a determination of the usefulness of each sample based on self-selected criteria. Although full specificity results are not available for samples which have not been purchased, the system is able to make cut-off calculations based on the non-purchased data to help highlight samples suitable for purchasing based on user defined criteria such as a maximum or minimum value.

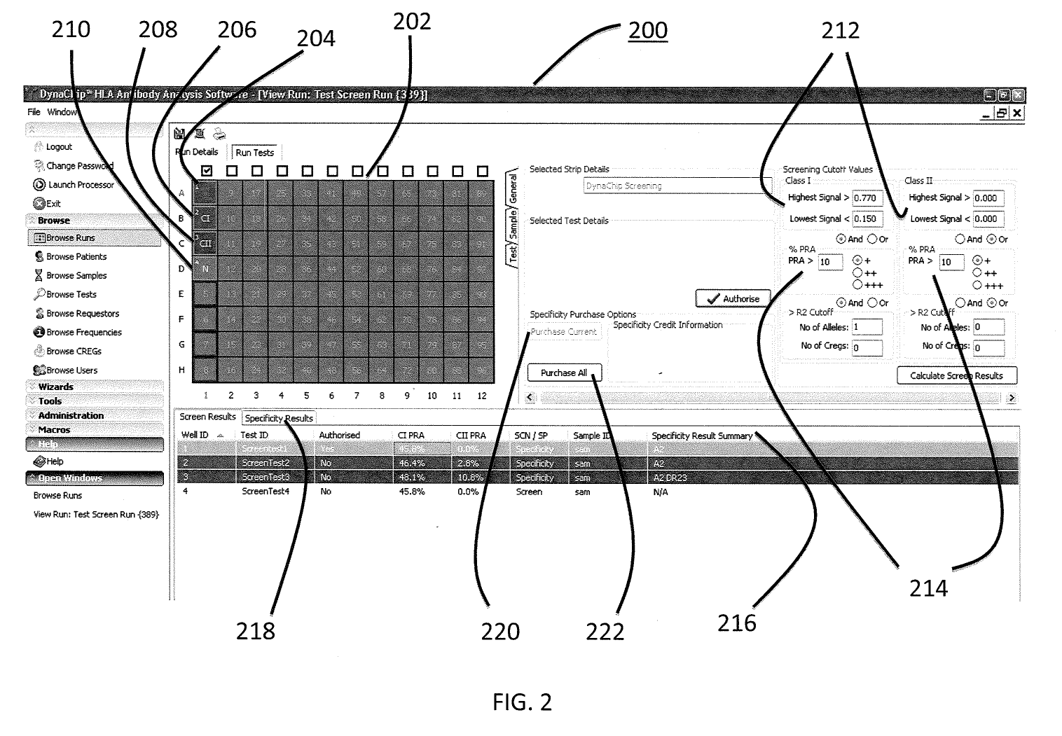

FIG. 2 depicts one embodiment of an interactive graphical user interface (“GUI”) 200 for screening data in a panel view 202. In the example shown, 4 samples have been tested in Well nos. 1-4 (204, 206, 208, 210), respectively. Well no. 4 210 did not produce a positive result during the initial screening stage and is therefore labeled with an “N.” The two columns on the right of the GUI 200 provide the preliminary diagnostic data such as the intensity of the signal 212 and the PRA value 214. Well nos. 1 to 3 (204, 206, 208) have been purchased using specificity credits, and so specificity results are shown in the area ‘Specificity Result Summary’ 216 (e.g. “A2”), but Well no. 4 210 has not been purchased, and so “N/A” is shown. In addition, the tab ‘Specificity Results’ 218—which allows access to the full data and statistical calculations—is accessible for the selected Well no. 1 204, as it has been purchased; but this tab is not available when Well no. 4 210 is selected.

The ‘Purchase Current’ 220 and ‘Purchase All’ buttons 222 increment a credit counter that tallies the amount of specificity credits available to the user and provide the full information for the customer to be able to view and interpret. Credits may be supplied via CDs with unique codes or by transmission over the Internet.

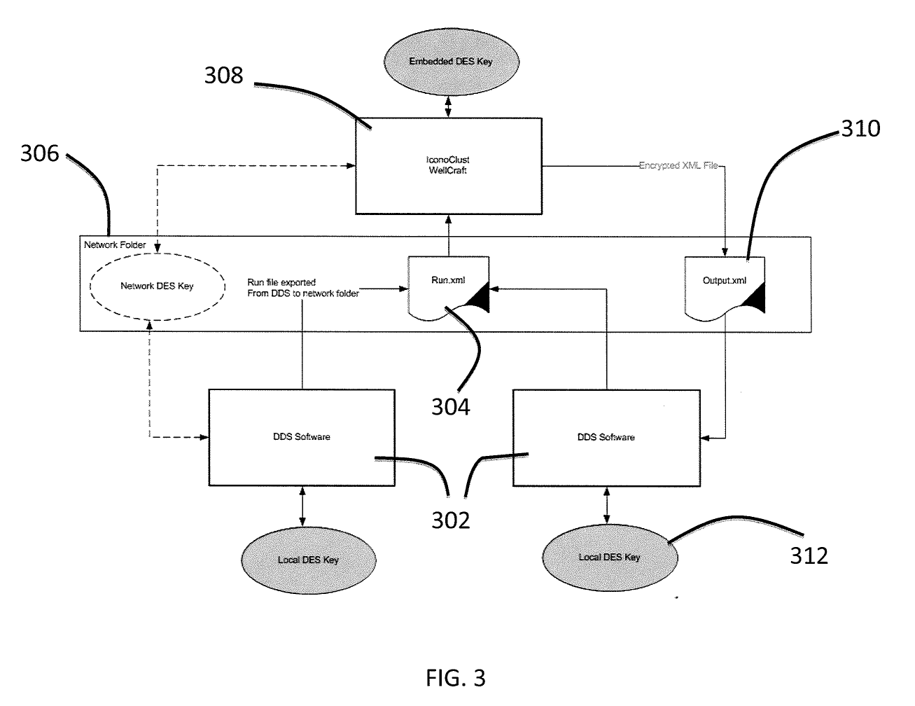

In a purchase-type embodiment where the specificity data must be purchased, the specificity data may be encrypted to separate and protect the data. In one embodiment, illustrated by the workflow in FIG. 3, a software architecture process for encrypting and decrypting the specificity data is provided. DDS software 302 first exports an extensible markup language (“XML”) file 304 for the panel test to a network folder 306. Once the array processor completes the assay, the file 304 is passed to image analysis software 308 to analyze the results of each sample in the panel. In this particular embodiment, Iconoclust/Wellcraft image software is used (Iconoclust/Wellcraft; Jena, Germany). The image software 308 then provides an output XML file 310 that is encrypted with a DES key 312 and is read by the DDS software 302 into a database (not shown). Based on the output XML file 310, the DDS software 302 performs the specificity analysis and reports screening results to the user. Once the user chooses which samples to retrieve specificity results for, the DES key 312 decrements the credit counter and decrypts the portion of XML data from the XML file 310 that corresponds to the specificity data purchased by the user. The decrypted XML data is then stored in the database, and the DDS software 302 displays the specificity results to the user (see FIG. 2). FIG. 3 also illustrates a method for encrypting and decrypting the data, as evident by the aforementioned steps.

One skilled in the art will appreciate that the system described above is not limited to antibody analysis, but is useful for any laboratory diagnostic device where an initial summary of results is useful to more efficiently analyze the detailed results. The system is particularly effective in a high volume, high throughput system such as sequence-based typing (“SBT”), where it is desired to eliminate further testing of negative samples.

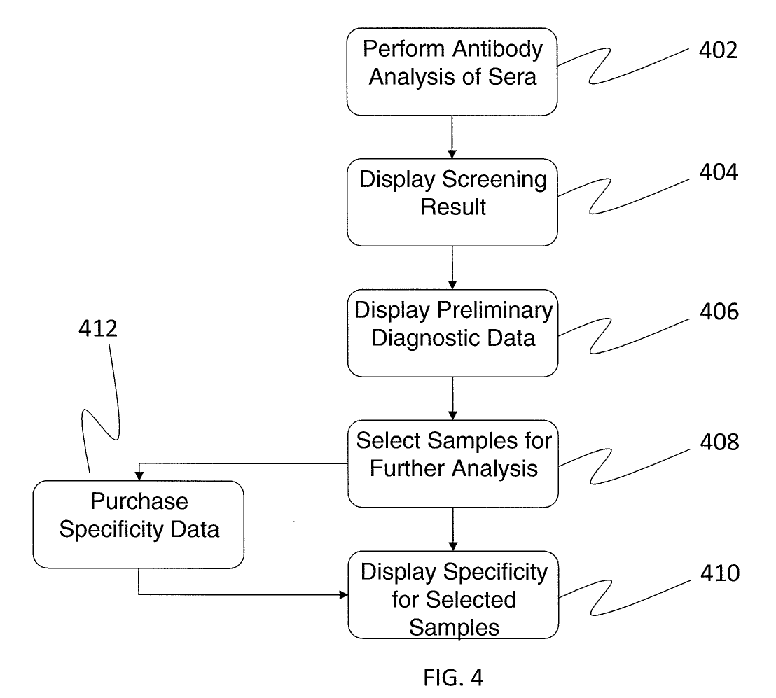

In one embodiment, the system described above for separating and screening preliminary diagnostic data may be embodied in a method of the same design, incorporating the elements and features of the system into the process described below and illustrated in FIG. 4. In the first step 402, an antibody analysis is completed of sample sera on a protein panel, which includes a complete specificity analysis. A screening result is then displayed for each sample in step 404, to indicate whether the screening result is positive or negative. Next, the preliminary diagnostic data is displayed for the positive results in step 406. In step 408, the user can select any number of samples for further analysis based on the information from the preliminary diagnostic data. Depending on the particular setup of the system, the user can either immediately view the specificity for the selected samples (step 410), or may have to purchase the complete specificity data for each desired sample, as described in step 412. As indicated above in one embodiment, the user first purchases specificity credits that are redeemed for specificity data.

The methods also relate to computer programs capable of being used in systems and methods for separating and screening preliminary diagnostic data. In particular, the methods relate to computer storage media comprising executable computer code, wherein the executable code is capable of displaying the results of the antibody analysis.

The methods may be implemented using hardware, software or a combination thereof and may be implemented in a computer system or other processing system. Various software implementations are described in terms of this exemplary computer system. After reading this description, it will become apparent to a person skilled in the relevant art how to implement the invention using other computer systems and/or computer architectures.

The computer where the system resides may also comprise a main memory, a random access memory (RAM), and, optionally a secondary memory. In the computer used to implement the method and programs of the present invention, storage for the programs is provided by the main memory and/or the secondary memory.

Examples of secondary memories include, but are not limited to, for example, a hard disk drive and/or a removable storage drive, representing a floppy disk drive, a magnetic tape drive, a compact disk drive, a DVD drive, a flash drive, etc. The removable storage drive may read from and/or write to a removable storage unit in a well-known manner.

Removable storage unit, also called a program storage device or a computer program product, represents a floppy disk, magnetic tape, compact disk, a DVD a flash drive, etc. As will be appreciated, the removable storage unit may also comprise a computer usable storage medium having stored therein computer software (programs) and/or data.

Computer programs can be stored in main memory and/or the secondary memory. Such computer programs include, for example, computer programs corresponding to the applications. These computer programs, when executed in their respective computers, enable the processors in those computers to perform the methods and features of the present system. Accordingly, such computer programs represent controllers of their respective computers.

Finally, it should be understood that processes and techniques described herein are not inherently related to any particular apparatus and may be implemented by any suitable combination of components. Further, various types of general purpose devices may be used in accordance with the teachings described herein. It may also prove advantageous to construct specialized apparatus to perform the method steps described herein. The present invention has been described in relation to particular examples, which are intended in all respects to be illustrative rather than restrictive. Those skilled in the art will appreciate that many different combinations of hardware, software, and firmware will be suitable for practicing the present invention. For example, the described software may be implemented in a wide variety of programming or scripting languages, such as Assembler, C/C++, perl, shell, PHP, Java, etc.

One non-limiting example of an apparatus that may be used with the processes and techniques described herein is the DYNACHIP™ Antibody Analysis System (Life Technologies, Carlsbad, Calif.). The DYNACHIP™ System is designed for the automated processing of serum samples. This instrument is capable of performing all steps of an assay from dispensing, incubation, and washing to image detection and results analysis. The instrument enables fully automated microarray processing in a convenient and accessible format.

The DYNACHIP™ System is based on a unique chip format which allows for increased multiplexing capabilities. Purified proteins are deposited onto the surface of a chip. In some embodiments the surface chemistry is such that the proteins are attached to the chip without the need for additional immobilisation substances. Individual chips are affixed to the bottom of an 8-well strip. The 8-well strips are inserted into a 96-well holding frame resembling an ELISA plate. The use of the 96 well ELISA format in the DYNACHIP™ System is convenient for standardized manipulation but other geometries may be used. Each chip is spotted with multiple proteins which allows for panels of class I and class II proteins for simultaneous analysis of one serum sample in a single well. In some embodiments, up to about 79 proteins may be spotted, up to about 89 proteins may be spotted, up to about 121 proteins may be spotted, up to about 139 proteins may be spotted, up to about 170 proteins may be spotted or up to about 200 proteins may be spotted.

A single DYNACHIP™ may be spotted with multiple class I and class II proteins from single donors. Positive controls on the chip would include Biotin, Human IgG and anti-Human IgG. A negative reading for a Biotin or Human IgG control spot may indicate that the detection antibody or substrate has not been added. A positive result for the anti-Human IgG spot indicates that all of the necessary reagents have been added. A negative result from the anti-Human IgG along with positive results from the other positive controls would indicate that detection antibody was added but not a serum sample. A chip would also contain negative control spots.

Reagents for use in an analysis would include one or more wash buffers, one or more substrate solutions, one or more sample diluents, one or more detection antibodies and one or more positive controls.

Proteins for spotting on the chips may be single donor proteins isolated from a single individual. HLA antigens are co-expressed membrane bound proteins having a trans-membrane tail. The proteins may be isolated from platelets of cell lines. In some embodiments the HLA antigens may be purified by affinity chromatography using class I and class II specific monoclonal antibodies. In some embodiments the HLA antigens are bound to the chip without the use of immobilization substances or protein linker molecules.

The 8-well strips holding the chips are loaded into a frame within the apparatus. In some embodiments the strips are provided in a bar coded package. These bar codes may be scanned into the computer during loading of the strips. The frame facilitates x-axis and y-axis movement of the chips so that reagents may be added and removed from individual wells and the wells can be imaged by the camera. The apparatus contains devices well known in the art for dispensing and aspirating reagents and containers for the storage of reagents and waste.

The apparatus may be covered to prevent dust or other contaminants from interfering with the assay. In some embodiments there may be sensors to detect the covers and prevent operation of the apparatus if the covers are not in place.

Once processing of the chips is complete, images of individual chips may be obtained by the use of a camera. To facilitate image capture, the chip may be illuminated. The light used for illuminating the chip may be white light or light of a specific wavelength such as red light, green light, blue light, yellow light etc.

In some embodiments, the apparatus facilitates automated processing of samples. An example of the steps involved in a typical sample run are:

1) Sample preparation, including dilution of the sample in the individual wells, followed by placing the sample strips in the processor and starting the analysis program,
2) Binding of antibodies to HLA-antigens to the chip followed by a wash step,
3) Binding of detection antibodies followed by a wash step and the addition of substrate,
4) Automated image capture if individual chips,
5) Integrated image data analysis and interpretation.

### Example 1

The processes and techniques described herein offer an automated workflow compared to bead-based and ELISA methods (Table 1). Automated processing eliminates manual pipetting and incubation steps, allowing the user to gain valuable lab time. After the initial setup of the processor, the user may be free to work on other projects.

TABLE 1
Comparison of workflows of the exemplary automated
DYNACHIP ™ system and manual antibody analysis methods.

| | Multiplex class I and class II analysis | | Single class I and |
|---|---|---|---|
| | DYNACHIP ™ | LUMINEX ® | class II analysis |
| | System | Platform | ELISA |
| I | Prepare serum samples and assay reagents | | |
| | Automated | | |
| | processing | Manual processing | |
| II | Load and start | Binding reaction | Prewet wells |
| | DYNACHIP ™ | | |
| | Processor | Incubate 30 min | Binding reaction |
| | Block | Spin | Incubate 30-40 min |
| | Wash | Wash 3x | Wash 3x |
| | Transfer sample | Add detection Ab | Add 2nd Ab |
| | Incubate chip | Incubate 30 min | Incubate 30-40 min |
| | Wash | Wash 3x | Wash 3x |
| | Detect | (Transfer to plate) | Add substrate |
| | Incubate | Add detection buffer | Incubate 30 min |
| | Wash | Wash | Add stop solution |
| | Add substrate | Load on reader | Take OD reading |
| | Image | | |

| | |
|---|---|
| III | Data analysis |

### Example 2

The processes and techniques described herein may allow the user to conserve precious serum samples (Table 2). When working with limited patient samples, it is important to conserve serum for future testing. The exemplary DYNACHIP™ system is designed to use as little as 8 μl of serum for the entire test, which includes class I and class II analysis.

TABLE 2
Comparison of recommended serum sample
volumes for antibody analysis methods.

| | DYNACHIP ™ | | LUMINEX ® | | Flow |
|---|---|---|---|---|---|
| Assay | System | ELISA | assay | | Cytometry |
| Class I and | | 24 μl | 20-40 | μl | 20 μl |
| II screen | | | | | |
| Class I | | 550 μl | 12.5-20 | μl | 20 μl |
| ID/PRA | | | | | |
| Class II | | 425 μl | 12.5-20 | μl | 20 μl |
| ID/PRA | | | | | |
| Serum | | | | | |
| sample total | 8 μl | 999 μl | 45-80 | μl | 60 μl |

### Example 3

The exemplary DYNACHIP™ system provides an automated alternative to ELISA for antibody analysis and provides comparable performance. In one study, 298 clinical samples were analyzed using the DYNACHIP™ method and a standard ELISA method. The results from comparing the two methods show a high level of concordance (Table 3).

TABLE 3
Comparison of the DYNACHIP ™ system with ELISA

| | | | | Overall |
|---|---|---|---|---|
| | | DYNACHIP ™ | DYNACHIP ™ | concor- |
| | | positive | negative | dance |
| Class | ELISA positive | 185 | 19 | 87.6% |
| I | ELISA negative | 18 | 76 | |
| Class | ELISA positive | 151 | 27 | 87.6% |
| II | ELISA negative | 10 | 110 | |

### Example 4

The performance of the exemplary DYNACHIP™ system was compared between two clinical laboratories using 94 identical serum samples. The results show that the DYNACHIP™ system provides similar performance results from lab to lab (Table 4).

TABLE 4
Antibody analysis between two laboratories with
the exemplary DYNACHIP ™ system.

| Antibody class | Overall concordance | P value |
|---|---|---|
| Class I | 88.29% | 0.3383 |
| Class II | 92.55% | 0.8413 |

### Example 5

Antibody specificity data using the exemplary DYNACHIP™ and LUMINEX® methods were compared for both sera and known cell types, with confirmation from flow cytometry crossmatch analysis. Three serum samples and six cell types were analyzed, for a total of 18 crossmatches (Tables 5 and 6).

TABLE 5
Comparison of antibody specificites detected by the exemplary
DYNACHIP ™ system and the LUMINEX ® platform,
with confirmation by flow cytometry crossmatch.

| | | DYNACHIP ™ | | LUMINEX ® | |
|---|---|---|---|---|---|
| | | Specificities | | specificities | |
| | | | Not | | Not |
| | | Confirmed | confirmed | Confirmed | confirmed |
| Flow | Positive | 8 | 5 | 11 | 2 |
| crossmatch | Negative | 5 | 0 | 2 | 3 |

TABLE 6
Comparative data from two samples showing the positive
correlation of data from the exemplary DYNACHIP ™ analysis
with flow cytometry crossmatch analysis.

| | | | Actual | Flow |
|---|---|---|---|---|
| | DYNACHIP ™ | LUMINEX ® | channel | crossmatch |
| Cells | specificities | specificities | shift | results* |
| Cell I: A3, | B52, B35, B70 | B62, B63, B75, B53, | 50 | Negative |
| A24, B8, | (71, 72), B17, | B35, B71, B78, B72, | | |
| B27, Cw2.7 | (57) | B52, B51, B58, B8, | | |
| | | B7, B65, B42, B41, | | |
| | | B18, B81, B55, B54 | | |
| | | B82 | | |
| Cell II: A31, | B12 (44, 45), | B44, B45, B60, B61, | 30 | Negative |
| A32, B62, | B60, B61, | B40, B41, B49, B50, | | |
| B35, Cw3 | (41, 49) | B13, B35, B62 | | |

*Positive cutoff equals 60-channel shift

### Example 6

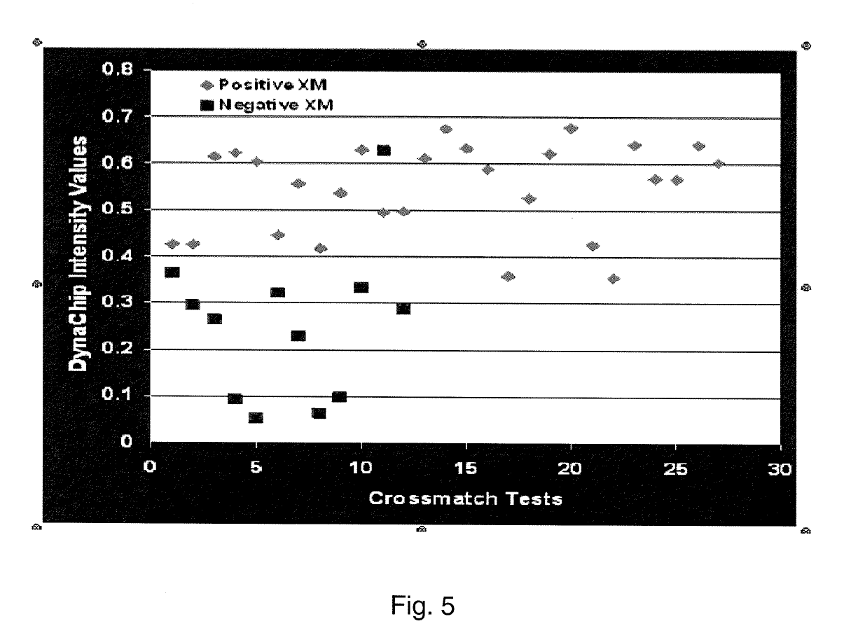

Results of 39 CDC-XMs were compared with those of DYNACHIP™ tests, the results are shown in FIG. 5. Closely matched phenotypes yielded mean DYNACHIP™ intensity values for positive and negative CDC-XMs of 0.547 and 0.252, respectively (p=0.00003).

### Example 7

DYNACHIP™ results were compared to those obtained by flow cytometry and Lunimex® in single donor or single antigen formats (Table 7).

TABLE 7
Antibody identification data comparing DYNACHIP ™ results with those
obtained by flow cytometry and LUMINEX ® in single donor or single antigen
formats for selected proficiency testing specimens (class I results only).

| | | | TP- | | TP- | | | |
|---|---|---|---|---|---|---|---|---|
| | | | LMX | 1Lamp- | LMX | TP-LMX | DC- | DC AB- |
| Sample | CL-AB¹ | FC-PRA¹ | SAB¹ | SAB¹ | PRA¹ | SDB¹ | PRA¹ | ID¹ |
| AB-41 | A2² | 79 | 15250 | 5274 | 68 | 7910 | 64 | X |
| | A68 | | 19019 | 7023 | | 10114 | | X |
| | A69 | | 17276 | 5779 | | — | | — |
| | A24 | | 11654 | 3473 | | 5145 | | X |
| | A33 | | 6856 | 2561 | | 4501 | | X |
| | A34 | | 9350 | 2368 | | 6153 | | X |
| | A66 | | 5626 | 1325 | | 2575 | | 0 |
| | A23 | | 5385 | 1550 | | 5485 | | X |
| | A25 | | 5745 | 1540 | | 2670 | | X |
| | A26 | | 3955 | 1125 | | 3016 | | X |
| | B18 | | CW10- | B18 | Cw10- | 5663 | | 0 |
| | | | 2808 | | 1935 | | | |
| AB-42 | A2 | 92 | 13242 | 11215 | 88 | 12858 | 75 | X |
| | A68 | | 14794 | 12558 | | 9943 | | X |
| | A69 | | 14661 | 12484 | | — | | — |
| | A3 | | 3363 | 2334 | | 4415 | | 0 |
| | A11 | | 2715 | 1924 | | <1000 | | 0 |
| | A24 | | 8143 | 5529 | | 6741 | | X |
| | A23 | | 4982 | 3335 | | 7261 | | X |
| | A33 | | 6856 | <1000 | | 2752 | | 0 |
| | A74 | | 2940 | 2129 | | 1938 | | X |
| | B7 | | 5712 | 4035 | | 7542 | | X |
| | B27 | | 4208 | 2810 | | 6487 | | X |
| | B57 | | 13229 | 10132 | | 8170 | | X |
| | B58 | | 13526 | 10645 | | 4533 | | X |
| | B81 | | 5146 | 3586 | | | | 0 |
| AB-43 | A2 | 97 | 17026 | 9249 | 96 | 15413 | 97 | X |
| | A68 | | 16903 | 8841 | | 10579 | | X |
| | A69 | | 17455 | 9447 | | — | | — |
| | A23 | | 6266 | 2422 | | 8356 | | X |
| | A24 | | 1163 | 4877 | | 9057 | | X |
| | A30 | | 9918 | 3653 | | 8005 | | X |
| | A3 | | 8699 | 3925 | | 7848 | | X |
| | A74 | | 6788 | 2908 | | 3930 | | X |
| | A11 | | 6746 | 3213 | | 5922 | | X |
| | A31 | | 6569 | 2860 | | 8102 | | X |
| | A32 | | 6505 | 2818 | | 6465 | | X |
| | A25 | | 6144 | 2361 | | 4716 | | X |
| | A33 | | 5936 | 2385 | | 5963 | | X |
| | A34 | | 5656 | 2724 | | 9142 | | X |
| | A80 | | 3981 | 0 | | 3716 | | 0 |
| | A66 | | 3885 | 0 | | 4189 | | 0 |
| | B57 | | 14418 | 6978 | | 11581 | | X |
| | B58 | | 14254 | 7264 | | 7281 | | X |
| | B46 | | 8453 | 4150 | | 5328 | | 0 |
| | B75 | | 7611 | 3305 | | 3414 | | 0 |
| | B63 | | 7092 | 2922 | | 11634 | | 0 |

¹CL-AB = Class I specificity; FC-PRA = flow cytometry PRA; TP-LMX SAB = Tepnel single antigen bead; 1 Lamb SAB = One Lambda single antigen beads; TP-SDB LMX = Tepnel single Donor beads; DC-PRA = DYNACHIP ™ PRA; DC-AB-ID = DYNACHIP ™ antibody identification; bold = AHG positive antibodies; underline = positive crossmatches.
²Specificities determined by One Lambda Flow Single Antigen Beads.
³Specificities <1000 for any LUMINEX ® system were not recorded as they have been determined in our laboratory to be inconsistent in reporducibility.
⁴“—” = no reagent for that specificity on panel.
⁵“0” - No specificity detected.

Although various representative embodiments of this invention have been described above with a certain degree of particularity, those skilled in the art could make numerous alterations to the disclosed embodiments without departing from the spirit or scope of the inventive subject matter set forth in the specification and claims. In methodologies directly or indirectly set forth herein, various steps and operations are described in one possible order of operation, but those skilled in the art will recognize that steps and operations may be rearranged, replaced, or eliminated without necessarily departing from the spirit and scope of the present invention. Also, various aspects and/or components of the described embodiments may be used singly or in any combination in the computerized storage system for capturing, classifying and linking collaboratively-captured media. It is intended that all matter contained in the above description or shown in the accompanying drawings shall be interpreted as illustrative only and not limiting.

What is claimed is:

1. A method for selecting one or more samples for displaying complete antibody specificity analysis data, the method comprising:
receiving complete specificity analysis data;
displaying an initial screening result for each sample, wherein the screening result is negative or positive, and wherein the positive sample includes preliminary diagnostic data, the preliminary diagnostic data being derived from the specificity analysis;
selecting at least one of the positive samples to display complete specificity analysis data, wherein the selection is based upon the preliminary diagnostic data; and
displaying the complete specificity analysis data for the selected samples.

2. The method of claim 1, further comprising requiring that the displaying of the complete specificity analysis data be purchased.

3. The method of claim 2, further comprising encrypting the complete specificity analysis data.

4. The method of claim 3, further comprising decrypting the complete specificity analysis data of the selected positive samples once the selected positive samples are purchased.

5. The method of claim 1, further comprising selecting a panel reactive antibody (“PRA”) value as the preliminary diagnostic data.

6. The method of claim 5, further comprising selecting an intensity value as the preliminary diagnostic data.

7. The method of claim 6, further comprising determining a desired range of PRA value and intensity value for displaying during the initial screening result.

8. The method of claim 7, further comprising customizing the initial screening result to recommend a positive sample for selection based upon whether the values of the preliminary diagnostic data fall within the desired range.

9. A system for selecting one or more samples for displaying complete antibody specificity analysis data, the system comprising:
receiving means for receiving complete specificity analysis data;
display means for displaying a screening result for each sample, the screening result being positive or negative, and wherein the positive sample includes preliminary diagnostic data, the preliminary diagnostic data being derived from the specificity analysis;
selection means for selecting at least one of the positive samples to display complete specificity analysis data, wherein the selection is based upon the preliminary diagnostic data; and;
display means for displaying the complete specificity analysis data for the selected samples.

10. The system of claim 9, further comprising purchasing means for requiring that the displaying of the complete specificity analysis data be purchased.

11. The system of claim 10, further comprising encryption means for encrypting the complete specificity analysis data.

12. The system of claim 11, further comprising decryption means for decrypting the complete specificity analysis data of the selected positive samples once the selected positive samples are purchased.

13. The system of claim 9, wherein the preliminary diagnostic data includes a panel reactive antibody (“PRA”) value.

14. The system of claim 13, wherein the preliminary diagnostic data further includes an intensity value.

15. The system of claim 14, wherein the selection means for selecting preliminary diagnostic data further comprises determining a desired range of PRA value and intensity value for displaying during the initial screening result.

16. The system of claim 15, further comprising customization means for customizing the initial screening result to recommend a positive sample for selection based upon whether the values of the preliminary diagnostic data fall within the desired range.

17. A computer program product embodied on a computer-readable medium comprising computer code for selecting one or more samples for displaying complete antibody specificity analysis data, wherein the computer code is operable for:
receiving a complete specificity analysis;
determining a screening result for each sample, the screening result being positive or negative, and wherein the positive sample includes preliminary diagnostic data, the preliminary diagnostic data being derived from the specificity analysis;
displaying an interactive screening report to a user, wherein a user can select at least one of the positive samples for displaying complete specificity analysis data; and
displaying the complete specificity analysis data for the selected samples.

18. The computer program product of claim 17, wherein the computer code is further operable for requiring that the displaying of the complete specificity analysis data be purchased.

19. The computer program product of claim 18, wherein the computer code is further operable for encrypting the complete specificity analysis data.

20. The computer program product of claim 19, wherein the computer code is further operable for decrypting the complete specificity analysis data of the selected positive samples once the selected positive samples are purchased.
